(19) United States
(12) Patent Application Publication
(10) Pub. No.: US 2025/0303099 A1
(43) Pub. Date: Oct. 2, 2025

(54) RESPIRATORY SUPPORT APPARATUS

(71) Applicant: FUKUDA DENSHI CO., LTD., Tokyo (JP)

(72) Inventors: Naoto SHIRAISHI, Tokyo (JP); Ryotaro HASHIMOTO, Tokyo (JP)

(21) Appl. No.: 19/089,213

(22) Filed: Mar. 25, 2025

(30) Foreign Application Priority Data
Mar. 29, 2024 (JP) 2024-056586
Jul. 2, 2024 (JP) 2024-106830

(51) Int. Cl. A61M 16/10; A61M 16/00

(52) U.S. Cl. CPC A61M 16/1085; A61M 16/0003; A61M 16/0066

(57) ABSTRACT

A respiratory support apparatus of the present disclosure includes: a main body housing that includes an intake port and an exhaust port and forms a flow path of air from the intake port to the exhaust port; a blower that is disposed in the flow path and generates airflow to be delivered into a patient's airway; a humidifier that is disposed on a downstream side of the blower in the flow path, adds moisture to the airflow, and adjusts a temperature of the airflow; and a detector that is disposed on a side of the blower with respect to an intermediate position between the intake port and the blower in the flow path and detects the temperature of the airflow.

### CROSS REFERENCE TO RELATED APPLICATIONS

This application is entitled to and claims the benefit of Japanese Patent Application No. 2024-106830, filed on Jul. 2, 2024 and Japanese Patent Application No. 2024-56586, filed on Mar. 29, 2024, the disclosure of which including the specification, drawings and abstract is incorporated herein by reference in its entirety.

### TECHNICAL FIELD

The present disclosure relates to a respiratory support apparatus.

### BACKGROUND ART

Conventionally, a respiratory support apparatus that supplies airflow to a patient, such as a continuous positive airway pressure (CPAP) apparatus, has been known. Note that, the CPAP apparatus is an apparatus used for CPAP therapy (also referred to as a sleep apnea treatment apparatus). CPAP therapy is a treatment method that prevents apnea in a patient during sleep, who has symptoms of obstructive sleep apnea, by continuously supplying air to the airway of the patient to widen the airway.

A respiratory support apparatus of this type generally includes: a blower that generates airflow; a humidifier that adds moisture to the airflow; and a built-in board for controlling the blower and the humidifier, in a main body housing, and is configured to be capable of generating airflow suitable for widening a patient's airway (for example, see Japanese Patent Application Laid-Open No. 2023-071739).

Such a respiratory support apparatus is described in, for example, Japanese Patent Application Laid-Open No. 2023-071739.

Incidentally, in a respiratory support apparatus of this type, a temperature/humidity sensor is typically mounted to control the humidifier to appropriately adjust the temperature and humidity of airflow.

In the respiratory support apparatus according to the related art, such a temperature/humidity sensor is disposed, as a means for measuring the environmental temperature and the environmental humidity around the apparatus, in the vicinity of an intake port of a main body housing of the respiratory support apparatus. However, in the respiratory support apparatus according to the related art, it cannot be said that the disposition position of the temperature/humidity sensor has been sufficiently considered, and there is room for improvement.

In particular, in the respiratory support apparatus according to the related art, there has been a possibility that, when the respiratory support apparatus is in operation, the environmental temperature and the environmental humidity around the apparatus measured by the temperature/humidity sensor may be in a state of being significantly different from the temperature and humidity of airflow flowing into the humidifier, and that it may be difficult to optimally control the humidifier. In other words, there has been a possibility that the temperature and humidity of the airflow supplied from the humidifier to the patient may deviate from those of the airflow suitable for the patient's breathing.

The present invention has been made in consideration of the above-described challenges, and an object of the present invention is to provide a respiratory support apparatus capable of attempting optimization of the temperature and humidity control of airflow to be supplied to a patient.

### SUMMARY

A main aspect of the present invention for solving the above-described challenges is a respiratory support apparatus that includes:
a main body housing that includes an intake port and an exhaust port and forms a flow path of air from the intake port to the exhaust port;
a blower that is disposed in the flow path and generates airflow to be delivered into an airway of a patient;
a humidifier that is disposed on a downstream side of the blower in the flow path, adds moisture to the airflow, and adjusts a temperature of the airflow; and
a detector that is disposed on a side of the blower with respect to an intermediate position between the intake port and the blower in the flow path and detects the temperature of the airflow.

### BRIEF DESCRIPTION OF THE DRAWINGS

FIG. 1 is a diagram illustrating how a CPAP apparatus is attached to a patient;

FIG. 2A is a perspective view of the CPAP apparatus according to an embodiment of the present invention as seen from obliquely above;

FIG. 2B is a perspective view of the CPAP apparatus according to the embodiment of the present invention as viewed from obliquely above;

FIG. 3 is an exploded perspective view of the CPAP apparatus according to the embodiment of the present invention;

FIG. 4 is a schematic diagram illustrating a route of airflow;

FIG. 5 is a block diagram illustrating the configuration of the CPAP apparatus according to the embodiment of the present invention;

FIG. 6 is a diagram illustrating a configuration of a flow path in a main body housing (flow path case) and a disposition configuration of a temperature/humidity sensor in the CPAP apparatus according to the embodiment of the present invention;

FIG. 7 is a flowchart illustrating an exemplary protection operation of a blower by a controller of the CPAP apparatus according to the embodiment of the present invention; and

FIG. 8 is a flowchart illustrating the exemplary protection operation of the blower by the controller of the CPAP apparatus according to the embodiment of the present invention.

### DESCRIPTION OF EMBODIMENTS

Hereinafter, a preferred embodiment of the present invention will be described in detail with reference to the accompanying drawings. Note that, in the present specification and drawings, components having substantially the same functions are denoted by the same reference signs, and redundant descriptions are omitted thereby.

Hereinafter, a CPAP apparatus (hereinafter, referred to as “CPAP apparatus 100”) will be described as a preferred application example of the respiratory support apparatus according to the present invention. Note that, the respiratory support apparatus according to the present invention can be applied not only to a CPAP apparatus but also to an adaptive servo ventilation (ASV) apparatus or a Nasal High Flow (NHF) apparatus.

### <1> Configuration of CPAP Apparatus 100 in Embodiment

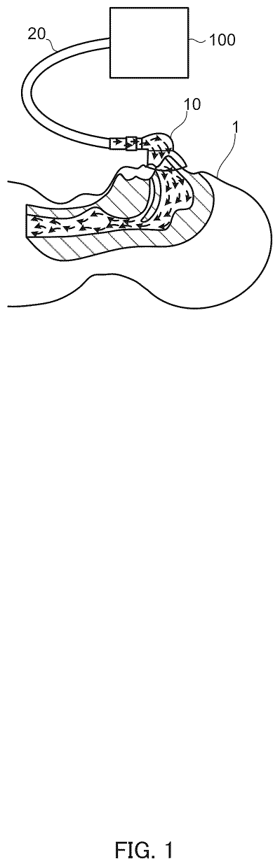

FIG. 1 is a diagram illustrating how CPAP apparatus 100 is attached to patient 1. As illustrated in FIG. 1, CPAP apparatus 100 includes mask 10 and tube 20, and an apparatus main body of CPAP apparatus 100 is connected to mask 10, which is put on the face of patient 1 with sleep apnea syndrome, via tube 20, and delivers positive pressure airflow for expanding the upper airway of patient 1 to the upper airway.

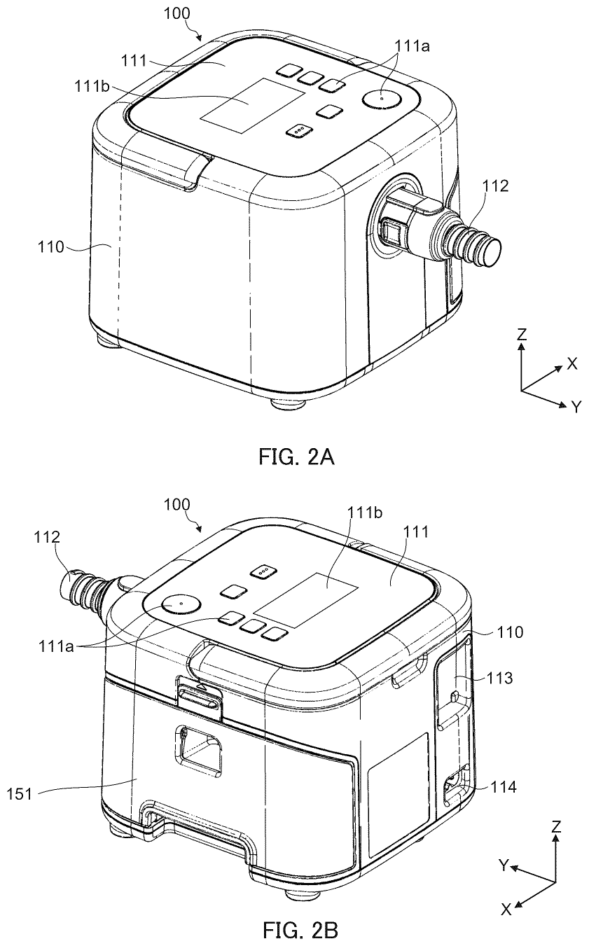

FIGS. 2A and 2B are perspective views of CPAP apparatus 100 as seen from obliquely above. Here, in FIGS. 2A and 2B, the +Z direction indicates the upward direction of CPAP apparatus 100, and the −Z direction indicates the downward direction of CPAP apparatus 100. Further, the +Y direction indicates the frontward direction of CPAP apparatus 100, and the −Y direction indicates the rearward direction of CPAP apparatus 100. Further, the +X direction indicates the left direction of CPAP apparatus 100, and the −X direction indicates the right direction of CPAP apparatus 100.

As can be seen in FIG. 2A, tube connector 112 to which tube 20 (FIG. 1) is connected protrudes from the front side surface of accommodation case 110 of CPAP apparatus 100. Further, operation panel 111 is provided in an upper portion of accommodation case 110. Operation panel 111 is provided with operation inputter 111a including an operation button or the like and display 111b.

As can be seen in FIG. 2B, intake port 113 and power connector 114 are provided on the rear side surface of accommodation case 110. An AC power supply is inputted into power connector 114 via a power cable. Further, water tank 151 is detachably attached to the right side surface of accommodation case 110.

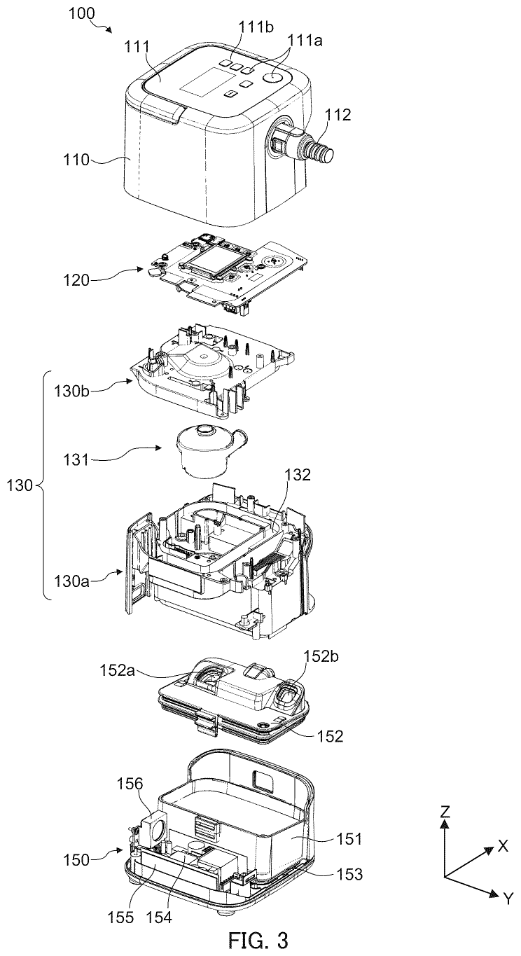

FIG. 3 is an exploded perspective view of CPAP apparatus 100.

CPAP apparatus 100 is configured to include accommodation case 110, circuit board 120, flow path case 130, and base 150.

Note that, in CPAP apparatus 100 according to the present embodiment, a main body housing of CPAP apparatus 100 is constituted by accommodation case 110, flow path case 130, and base 150 (hereinafter, the main body housing will also be referred to as “main body housing 100A”).

Accommodation case 110 has a rectangular tube shape, and accommodates circuit board 120, flow path case 130, and the like by being coupled to base 150 from above. Further, operation panel 111 is disposed in the upper portion of accommodation case 110.

As described above, accommodation case 110 includes tube connector 112, intake port 113, and power connector 114. Note that, tube connector 112 constitutes an exhaust port of main body housing 100A.

Circuit board 120 is provided with a central processing unit (CPU), various driver circuits, and the like.

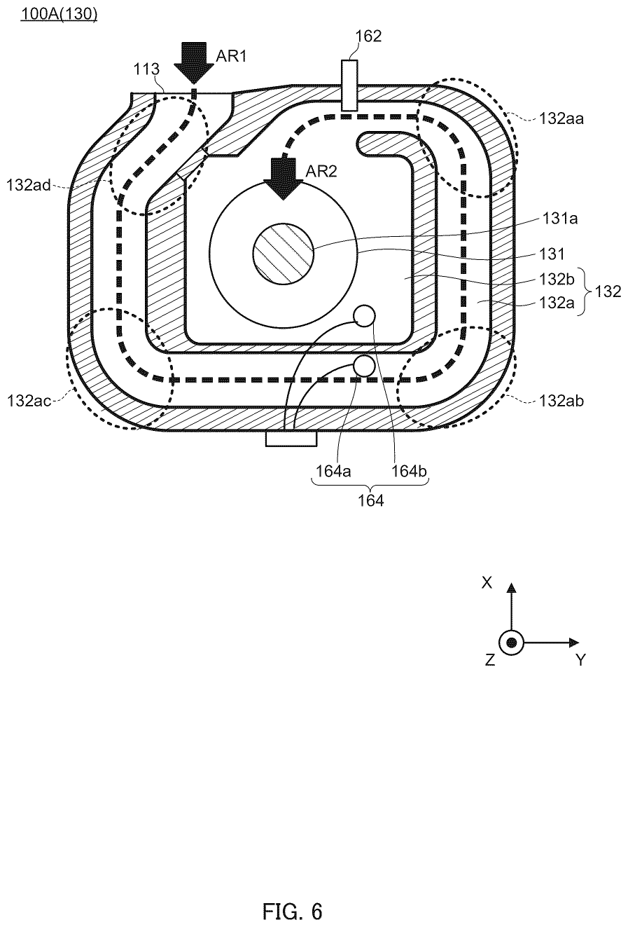

Flow path case 130 is configured by fitting lower case 130a and upper case 130b together. Blower 131 is disposed inside flow path case 130. In flow path case 130, flow path 132 (to be described later with reference to FIG. 6) is formed through which air sucked in through intake port 113 passes, and blower 131 is disposed in flow path 132. Note that, blower 131 gives energy to the air sucked in through intake port 113 and flowing through flow path 132, increases the pressure, and sends the air to a side of a humidifier constituted by water tank 151 or the like.

In base 150, water tank 151 is disposed which is detachable. In lid 152 of water tank 151, air inlet port 152a and air exhaust port 152b are formed. Air inlet port 152a communicates with flow path 132 in flow path case 130. Air exhaust port 152b communicates with tube connector 112.

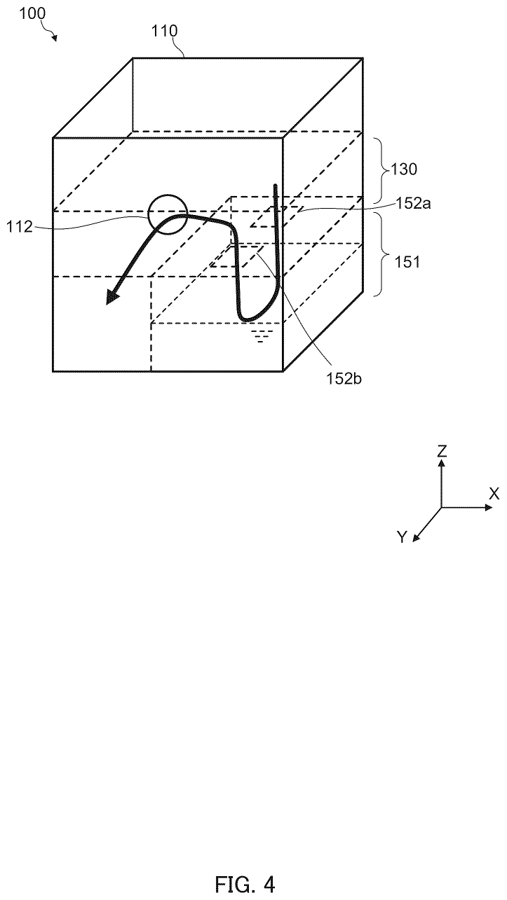

Thereby, as can be understood from the schematic diagram in FIG. 4, the airflow (the arrow in FIG. 4) passes through flow path 132 in flow path case 130, then enters water tank 151 through air inlet port 152a, is discharged from water tank 151 through air exhaust port 152b, and is supplied to patient 1 via tube connector 112.

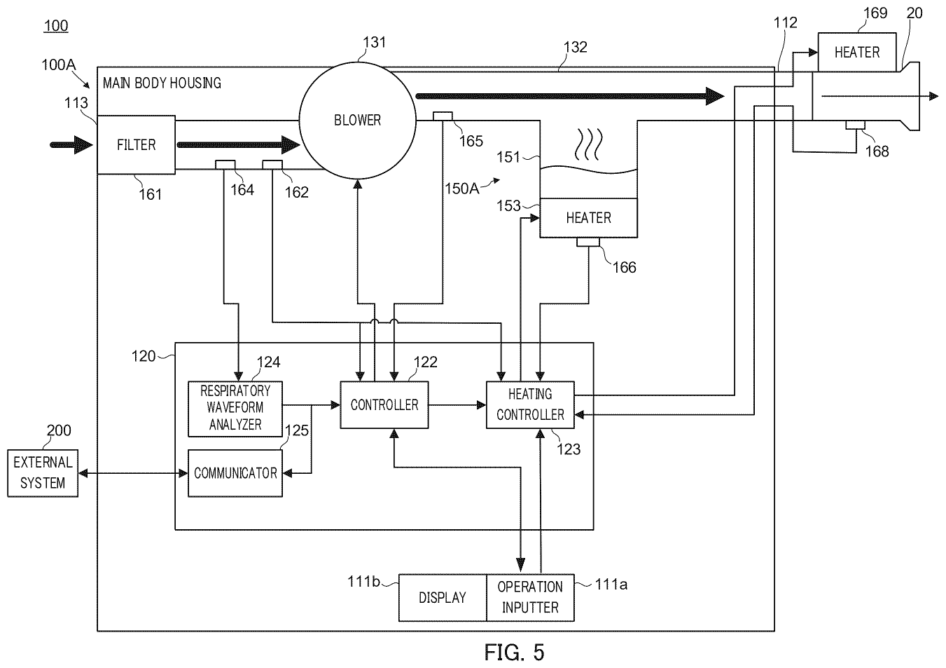

Heater 153 is provided on a side of the lower surface of water tank 151. The water in water tank 151 is heated by heater 153, resulting in a high humidity state in water tank 151. Thus, the airflow to be supplied to the patient is humidified in water tank 151. The drying of the airway of patient 1 due to the airflow is suppressed thereby. That is, in CPAP apparatus 100 according to the present embodiment, a humidifier that humidifies the airflow to be supplied to patient 1 is constituted by water tank 151, lid 152, and heater 153 (hereinafter, the humidifier will also be referred to as “humidifier 150A”) (see FIG. 5).

Further, AC/DC converter 154 is provided in base 150. AC/DC converter 154 receives an input of an external AC power supply from a power cord (not illustrated) connected to power connector 114 (FIG. 2B), converts the AC power supply into a DC power supply, and supplies the converted DC power supply to circuit board 120 and the like.

The lower side and both the left and right sides of a plurality of circuit components constituting AC/DC converter 154 are covered by sheet metal member 155 which has a U-shaped cross section cut in the XZ plane. Sheet metal member 155 extends in the Y direction. Fan 156 for cooling AC/DC converter 154 is provided on a side of one end of sheet metal member 155. Fan 156 is provided at a position facing AC/DC converter 154.

Thereby, AC/DC converter 154 is efficiently cooled by the wind of fan 156, which flows through a space covered by sheet metal member 155. Further, electromagnetic noise generated by AC/DC converter 154 is shielded by sheet metal member 155, thereby reducing the influence of the electromagnetic noise on other circuit boards and the like.

As described above, in CPAP apparatus 100 according to the present embodiment, the air sucked in through intake port 113 passes through flow path 132, blower 131, and humidifier 150A, and is supplied to patient 1 via tube connector 112.

FIG. 5 is a block diagram illustrating the configuration of CPAP apparatus 100.

In flow path 132 of CPAP apparatus 100, filter 161, temperature/humidity sensor 162, flow sensor 164, and pressure sensor 165 are provided in addition to blower 131. Further, temperature sensor 166 is attached to heater 153 that heats water tank 151. Note that, temperature/humidity sensor 162 corresponds to the “detector” of the present invention.

Circuit board 120 is provided with controller 122, heating controller 123, respiratory waveform analyzer 124, communicator 125, and the like. In other words, circuit components for implementing each function of controller 122, heating controller 123, respiratory waveform analyzer 124, and communicator 125 are mounted in circuit board 120.

Note that, controller 122, heating controller 123, and respiratory waveform analyzer 124 are each constituted by, for example, a microcomputer including a central processing unit (CPU), a read only memory (ROM), and a random access memory (RAM). The CPU reads a program corresponding to a processing content from the ROM, develops the program in the RAM, and cooperates with the developed program to implement each function of controller 122, heating controller 123, and respiratory waveform analyzer 124. Note that, controller 122, heating controller 123, and respiratory waveform analyzer 124 may be formed entirely or partially of a hard-wired circuit such as an application specific integrated circuit (ASIC) or a field-programmable gate array (FPGA).

When blower 131 operates, external air enters flow path 132 via intake port 113 and filter 161. The temperature and humidity of the air in flow path 132 are measured by temperature/humidity sensor 162, and the measured temperature and humidity are sent to controller 122 and heating controller 123. Further, a heating set value and a humidification set value (for example, a target temperature and a target humidity) from operation inputter 111a are inputted into heating controller 123, and temperature information of heater 153 from temperature sensor 166 is inputted into heating controller 123.

Heating controller 123 controls heater 153 based on information on the temperature and humidity measured by temperature/humidity sensor 162, the heating set value and the humidification set value from operation inputter 111a set by the user, and the temperature information of heater 153 from temperature sensor 166. For example, heating controller 123 controls heater 153 such that the temperature and humidity of the airflow to be supplied to patient 1 approach the heating set value and the humidification set value, respectively.

Further, temperature information from temperature sensor 168 provided in tube 20 is inputted into heating controller 123. Heating controller 123 controls heater 169 provided in tube 20 based on this temperature information to suppress condensation in tube 20.

Flow sensor 164 measures the flow rate of airflow passing through flow path 132. Flow sensor 164 is, for example, a differential pressure sensor, and measures the flow rate of airflow based on a pressure difference between two points of an upstream-side measurement point and a downstream-side measurement point, and sends the measurement result to respiratory waveform analyzer 124.

For example, respiratory waveform analyzer 124 acquires measurement data related to the flow rate of airflow from flow sensor 164, and detects the respiration flow (that is, the respiratory waveform) of patient 1 based on a temporal change in the flow rate of the airflow. Then, respiratory waveform analyzer 124 performs, for example, frequency analysis (for example, FFT analysis) or the like on the detected respiratory flow (respiratory waveform), and sends the analysis result (for example, signal intensity for each frequency) as respiration information to controller 122. Further, respiratory waveform analyzer 124 sends the respiration information to communicator 125, for example.

Pressure information on the pressure in flow path 132 measured by pressure sensor 165 is sent to controller 122. Further, pressure setting information (for example, target pressure) from operation inputter 111a is inputted into controller 122.

Controller 122 controls the pressure of the airflow to be supplied to patient 1 by controlling the rotation of blower 131 based on the pressure information measured by pressure sensor 165, the respiration information from respiratory waveform analyzer 124, and the pressure setting information from operation inputter 111a set by the user.

Further, controller 122 specifies the usage temperature of blower 131 based on the information on the temperature measured by, for example, temperature/humidity sensor 162, and limits of the operation (for example, decreases the output) of humidifier 150A or stops the operation of humidifier 150A to prevent deterioration of blower 131 in a case where the usage temperature exceeds a threshold temperature. Note that, for example, 50 degrees Celsius is set as the threshold temperature.

Communicator 125 performs communication with external system 200. For example, the respiration information obtained by respiratory waveform analyzer 124 is transmitted to external system 200 via communicator 125. Thereby, a medical professional who is at a location away from CPAP apparatus 100 can know the breathing state of patient 1, and can know, for example, that patient 1 is experiencing apnea.

### <2> Disposition Configuration of Temperature/Humidity Sensor

Next, details of a disposition configuration of temperature/humidity sensor 162 in CPAP apparatus 100 according to the present embodiment will be described. Note that, in general, the relative humidity is determined by the ratio of the amount of water vapor to the saturated water vapor amount at the temperature at that time, and thus, the temperature/humidity sensor has a configuration in which a temperature sensor and a humidity sensor are integrally disposed and the ambient temperature and the ambient humidity around the temperature/humidity sensor are measured simultaneously.

Note that, in the CPAP apparatus according to the related art, such a temperature/humidity sensor is disposed, as a means for measuring the environmental temperature and the environmental humidity around the apparatus, in the vicinity of the intake port of the main body housing of the respiratory support apparatus. In CPAP apparatuses in recent years, however, it has been considered to make the flow path from the intake port to the blower in the main body housing a relatively long path in order to reduce noise generated at the blower (see, for example, FIG. 6 to be described later). Further, it is also possible to reduce noise by disposing a sound-absorbing material on the route thereof and increasing the length thereof. In such an apparatus configuration, the temperature of airflow (that is, air that the blower sucks in and/or air that flows into the humidifier) may increase due to the thermal influence from a heating element in the CPAP apparatus when the airflow passes through the flow path in the CPAP apparatus. Examples of such a heating element include an AC/DC power supply and a humidifier which are built into the CPAP apparatus. When the CPAP apparatus is in continuous operation, the temperature of the main body housing of the CPAP apparatus increases due to the heat generation of the AC/DC power supply and/or the humidifier, and the temperature of airflow passing through the main body housing also increases correspondingly.

As a result, in the CPAP apparatus according to the related art, there has been a possibility that, when the CPAP apparatus is in operation, the environmental temperature and the environmental humidity around the apparatus measured by the temperature/humidity sensor may be in a state of deviating from the temperature and humidity of airflow flowing into the humidifier, and that it may be difficult to optimally control the humidifier. In other words, there has been a possibility that the temperature and humidity of the airflow supplied from the humidifier to the patient may deviate from those of the airflow suitable for the patient's breathing.

In addition, in the CPAP apparatus according to the related art, there has been a possibility that the lifespan of the blower may be shortened or the probability of failure of the blower may increase because the blower is continuously used in a state in which the usage temperature of the blower reaches an abnormal temperature due to an increase in the temperature of air sucked in by the blower.

Note that, the blower generally has an upper limit of the usage temperature (for example, approximately 50 degrees Celsius) (hereinafter also referred to as a heat-resistant temperature) according to the suction air as an apparatus specification. In a case where the blower is continuously used in a state exceeding the upper limit of such a heat-resistant temperature, the lifespan of the blower may be shortened or the probability of failure of the blower may increase.

However, in the CPAP apparatus according to the related art, it cannot be said that countermeasures against those mentioned above have been sufficiently considered, and there is room for improvement.

CPAP apparatus 100 according to the present embodiment employs a disposition configuration of temperature/humidity sensor 162 in consideration of the above-described challenges.

FIG. 6 is a diagram illustrating a configuration of flow path 132 in main body housing 100A (flow path case 130) and a disposition configuration of temperature/humidity sensor 162 in CPAP apparatus 100 according to the present embodiment.

Flow path 132 includes: guidance path 132a that guides air from intake port 113 to the position of blower 131; and blower disposition chamber 132b that is formed to be connected to the downstream end of guidance path 132a.

More specifically, blower disposition chamber 132b is a region in which blower 131 is disposed, and has, for example, a substantially rectangular shape in an XY plane view. Blower disposition chamber 132b is formed, in an XY plane view, by huge expansion of the flow path width in the ±Y direction from guidance path 132a. Note that, in the same manner, the cross-sectional area orthogonal to the flow direction of airflow is also hugely expanded. Blower 131 is disposed in blower disposition chamber 132b such that suction port 131a (the hatched region in FIG. 6) of blower 131 is located in the center of rectangular blower disposition chamber 132b in an XY plane view. Note that, CPAP apparatus 100 according to the present embodiment has a configuration in which blower 131 includes suction port 131a that opens in a direction (that is, the Z-axis direction) intersecting with the air guidance direction of guidance path 132a, from the viewpoint of securing a long flow path length of guidance path 132a. Blower disposition chamber 132b is formed to bend air coming from guidance path 132a in the direction intersecting with the guidance direction of guidance path 132a, and is formed to have a widened flow-path cross-sectional area.

Note that, the air (black arrow AR1) introduced through intake port 113 is sucked into suction port 131a of blower 131 (black arrow AR2) via guidance path 132a and blower disposition chamber 132b, and is sent to water tank 151 (humidifier 150A) via air inlet port 152a through an exhaust port (not illustrated) of blower 131. Note that, when air is sucked into suction port 131a of blower 131, the air is sucked in from the +Z direction toward the −Z direction.

Guidance path 132a includes at least one of bends 132aa to 132ad to guide air from intake port 113 to blower 131 such that the air largely detours in main body housing 100A. Guidance path 132a according to the present embodiment includes four bends 132aa to 132ad such that guidance path 132a passes through positions at four corners in main body housing 100A in a plan view. That is, guidance path 132a according to the present embodiment guides air such that the air circulates in main body housing 100A along the side walls in main body housing 100A, instead of guiding the air in a straight-line manner with the shortest distance from intake port 113 to the position of blower 131 in main body housing 100A. This is, as described above, to increase the length of flow path 132 from intake port 113 to the position of blower 131 and to reduce the degree to which noise generated at blower 131 leaks to the outside.

In such flow path 132, temperature/humidity sensor 162 is disposed at a position immediately before blower 131, and is at least disposed on a side of blower 131 with respect to an intermediate position between intake port 113 and blower 131. Specifically, temperature/humidity sensor 162 according to the present embodiment is disposed on the downstream side of bend 132aa on the most downstream side among four bends 132aa to 132ad. In other words, temperature/humidity sensor 162 according to the present embodiment is disposed on the direct upstream side of a connection position between guidance path 132a and blower disposition chamber 132b.

Further, when the disposition position of temperature/humidity sensor 162 is described in a positional relationship with flow sensor 164 (that is, a differential pressure sensor), temperature/humidity sensor 162 is disposed on the downstream side of upstream side-measurement point 164a of flow sensor 164. Note that, upstream side-measurement point 164a of flow sensor 164 is typically disposed by securing a sufficient distance from intake port 113 such that upstream side-measurement point 164a does not pick up turbulence in the vicinity of intake port 113 as measurement data. Upstream-side measurement point 164a is disposed, for example, on a side of blower 131 with respect to an intermediate position between intake port 113 and blower 131 in flow path 132. Further, in the present embodiment, downstream-side measurement point 164b of flow sensor 164 is disposed at a position in blower disposition chamber 132b, where the position is on the side opposite to the connection position between guidance path 132a and blower disposition chamber 132b (that is, in blower disposition chamber 132b, in the vicinity of the surface facing the connection position between guidance path 132a and blower disposition chamber 132b).

The reason why temperature/humidity sensor 162 is disposed at such a position in CPAP apparatus 100 according to the present embodiment is to implement a function of measuring the humidity and temperature of airflow before humidification and a function of monitoring the usage temperature of blower 131 with one temperature/humidity sensor 162. That is, as described above, when flow path 132 from intake port 113 to blower 131 in main body housing 100A is made long, airflow passing through main body housing 100A may be thermally affected by AC/DC converter 154, humidifier 150A, and/or the like which is/are built into CPAP apparatus 100, and the temperature of the airflow may increase during the flow thereof.

As a result, there is a possibility that, when the CPAP apparatus is in operation, the environmental temperature and the environmental humidity around the apparatus measured by the temperature/humidity sensor may be in a state of being significantly different from the temperature and humidity of airflow flowing into the humidifier, and that it may be difficult to optimally control the humidifier. In addition, the blower may be damaged since, although the usage temperature of the blower has reached an abnormal temperature due to an increase in the temperature of air sucked in by the blower, the blower is continuously used by regarding the environmental temperature around the apparatus as the usage temperature of the blower.

Given such a viewpoint, in CPAP apparatus 100 according to the present embodiment, the temperature and humidity of air after the introduction of the air through intake port 113 and a subsequent increase in the temperature of the air while the air is passing through flow path 132 are measured at a position immediately before blower 131 by temperature/humidity sensor 162. Since it is possible to accurately grasp the humidity and temperature of airflow immediately before entering humidifier 150A thereby, it is possible to conduct control of humidifier 150A with high accuracy in accordance with the target temperature and the target humidity of airflow to be delivered into a patient's airway. Further, since it is possible to accurately grasp the usage temperature of blower 131 thereby, it is also possible to prevent the lifespan of blower 131 from being shortened and to prevent the probability of failure of blower 131 from increasing.

Note that, the reason why temperature/humidity sensor 162 is disposed on the direct upstream side of the connection position between guidance path 132a and blower disposition chamber 132b in flow path 132 in the present embodiment is that the flow-path cross-sectional area of blower disposition chamber 132b is greatly expanded from guidance path 132a, and thus, the flow velocity of airflow increases on the direct upstream side of the connection position between guidance path 132a and blower disposition chamber 132b. That is, in general, the faster the flow velocity of airflow in the vicinity of the temperature/humidity sensor is, the more the measurement accuracy and responsiveness of the temperature/humidity sensor improve, and thus, by disposing temperature/humidity sensor 162 at this position in CPAP apparatus 100, it is possible to grasp the humidity and temperature of airflow in flow path 132 more accurately.

On the other hand, in a case where temperature/humidity sensor 162 is disposed on a side of a discharge port of blower 131, there is a high risk that temperature/humidity sensor 162 will be affected by humidifier 150A (water tank 151) to become unusable due to condensation when CPAP apparatus 100 is actually used. For this reason, temperature/humidity sensor 162 according to the present embodiment is disposed in front of blower 131.

Next, an exemplary operation of controller 122 for preventing the heat-resistant temperature of blower 131 from being exceeded will be described.

In CPAP apparatus 100 according to the present embodiment, controller 122 executes a protection operation to prevent the heat-resistant temperature of blower 131 from being exceeded.

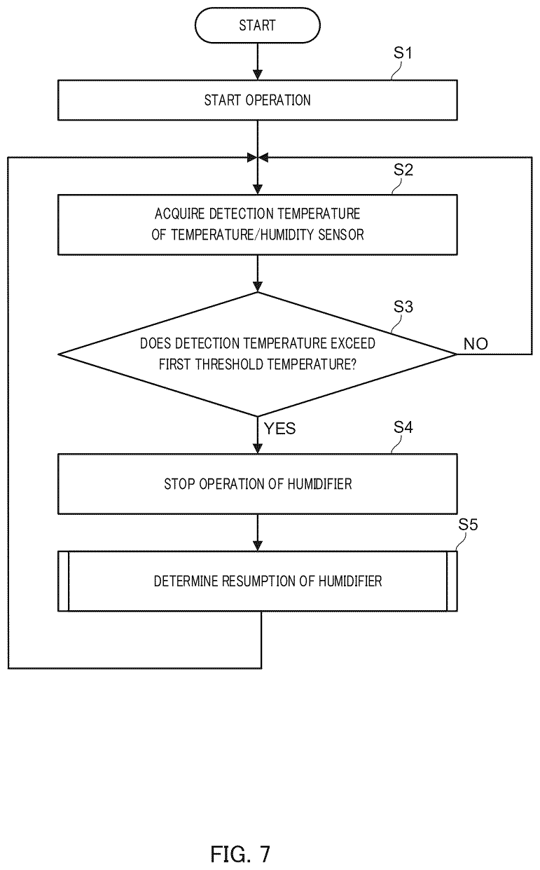

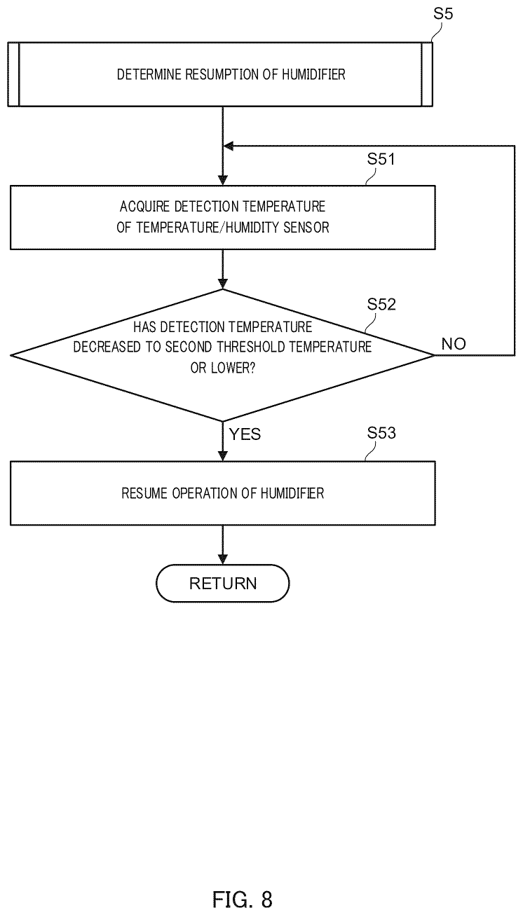

FIGS. 7 and 8 are flowcharts illustrating an exemplary protection operation of blower 131 by controller 122. The flowcharts in FIGS. 7 and 8 illustrate abnormality monitoring processing that controller 122 executes according to a program when CPAP apparatus 100 is activated. This processing is continuously executed while CPAP apparatus 100 is in operation, for example, while CPAP apparatus 100 is supplying airflow to a patient.
In step S1, controller 122 causes the normal operation of CPAP apparatus 100 to start by driving and controlling blower 131, heater 153, and the like.
In step S2, controller 122 acquires a temperature detected by temperature/humidity sensor 162.
In step S3, controller 122 determines whether the acquired detection temperature exceeds a first threshold temperature. Then, in a case where the detection temperature exceeds the first threshold temperature (for example, 50 degrees Celsius) (S3: YES), controller 122 advances the processing to step S4, whereas in a case where the detection temperature does not exceed the first threshold temperature (S3: NO), controller 122 returns to step S2 and continues the temperature monitoring processing.
In step S4, controller 122 stops the operation of humidifier 150A (that is, heater 153). Note that, at this time, controller 122 may stop heater 169 of tube 20, instead of stopping heater 153 of humidifier 150A or together with stopping heater 153 of humidifier 150A.
In step S5, controller 122 determines the timing for the resumption of the operation of humidifier 150A.
Step S5 includes sub-steps S51 to S53 as illustrated in FIG. 8.
In sub-step S51, controller 122 acquires the temperature detected by temperature/humidity sensor 162.
In sub-step S52, controller 122 determines whether the acquired detection temperature has decreased to a second threshold temperature or lower (the second temperature is a temperature equal to or lower than the first threshold temperature and is, for example, 40 degrees Celsius). Note that, in a case where the second threshold temperature is the same as or close to the first threshold temperature, humidifier 150A may be repeatedly turned on and off in a short cycle. Accordingly, the second threshold temperature is preferably a temperature lower than the first threshold temperature, and further a difference in temperature to some extent is preferably set therebetween. For example, a difference of five to ten degrees is provided between the second threshold temperature and the first threshold temperature.

In a case where the detection temperature has decreased to the second threshold temperature or lower (S52: YES), controller 122 advances the processing to sub-step S53, whereas in a case where the detection temperature has not decreased to the second threshold temperature or lower (S52: NO), controller 122 returns to sub-step S51 and continues the temperature monitoring processing.
In sub-step S53, controller 122 causes the operation of humidifier 150A to resume. Then, controller 122 returns to the processing in step S2 of the flowchart in FIG. 7 again, and continues the temperature monitoring processing.

As described above, controller 122 according to the present embodiment continuously monitors the usage temperature of blower 131 based on the temperature detected by temperature/humidity sensor 162. In addition, in a case where the usage temperature of blower 131 exceeds the first threshold temperature, controller 122 stops the operation of humidifier 150A to suppress a further increase in the usage temperature of blower 131. When the operation of humidifier 150A is stopped, the temperature of humidifier 150A decreases, and in addition, the heat generation of AC/DC converter 154 can also be suppressed, and thus, the temperature of main body housing 100A decreases, and the temperature of air sucked in by blower 131 also decreases. Thereby, it is possible to prevent a situation in which blower 131 is continuously used beyond the heat-resistant temperature.

Then, controller 122 causes humidifier 150A to operate again when the temperature detected by temperature/humidity sensor 162 has decreased to the second threshold temperature. Thereby, it is possible to continuously use CPAP apparatus 100 in a state in which the temperature of blower 131 is equal to or lower than the heat-resistant temperature.

### <3> Summary

As described above, CPAP apparatus 100 according to the present embodiment includes: main body housing 100A that includes intake port 113 and exhaust port 112 and forms flow path 132 of air from intake port 113 to exhaust port 112; blower 131 that is disposed in flow path 132 and generates airflow to be delivered into an airway of a patient; humidifier 150A that is disposed on a downstream side of blower 131 in flow path 132, adds moisture to the airflow, and adjusts a temperature of the airflow; and temperature/humidity sensor 162 that is disposed on a side of blower 131 with respect to an intermediate position between intake port 113 and blower 131 in flow path 132 and detects the temperature of the airflow.

Accordingly, CPAP apparatus 100 according to the present embodiment makes it possible to implement a function of accurately measuring the humidity and temperature of airflow before humidification and a function of monitoring the usage temperature of blower 131 with one temperature/humidity sensor 162.

Thereby, it is possible to appropriately control humidifier 150A such that airflow to be delivered from humidifier 150A to a patient becomes airflow suitable for the patient's breathing.

In addition, it is also possible to prevent the lifespan of blower 131 from being shortened and to prevent the probability of blower 131 from increasing thereby.

The present invention is not limited to the above embodiment, and is applicable to various modification aspects.

For example, in the above embodiment, an aspect in which accommodation case 110, flow path case 130, and base 150 constitute main body housing 100A of CPAP apparatus 100 has been indicated. However, these do not necessarily need to be separable in terms of realizing CPAP apparatus 100 according to the present invention.

Further, in the above embodiment, an aspect in which blower 131 increases the pressure of air flowing through flow path 132 has been indicated. However, in terms of realizing CPAP apparatus 100 according to the present invention, blower 131 does not necessarily need to discharge high-pressure airflow, and may discharge low-pressure airflow, such as that of a so-called fan.

Any of the embodiment described above is only illustration of an exemplary embodiment for implementing the present invention, and the technical scope of the present invention should not be construed limitedly thereby. That is, the present invention can be implemented in various forms without departing from the spirit or main features thereof.

At least following matters will be apparent from the description of the present specification and the accompanying drawings.

The present specification discloses a respiratory support apparatus including: a main body housing that includes an intake port and an exhaust port and forms a flow path of air from the intake port to the exhaust port; a blower that is disposed in the flow path and generates airflow to be delivered into an airway of a patient; a humidifier that is disposed on a downstream side of the blower in the flow path, adds moisture to the airflow, and adjusts a temperature of the airflow; and a detector that is disposed on a side of the blower with respect to an intermediate position between the intake port and the blower in the flow path and detects the temperature of the airflow.

Thereby, it is possible to appropriately control the humidifier such that airflow to be delivered from the humidifier to a patient becomes airflow suitable for the patient's breathing. In addition, it is also possible to prevent the lifespan of the blower from being shortened and to prevent the probability of failure of the blower from increasing thereby.

In the respiratory support apparatus, the flow path preferably includes a bend that guides the air from the intake port to a position of the blower such that the air largely detours in the main body housing.

Thereby, it is possible to reduce the degree to which noise generated at the blower leaks to the outside.

Further, in the respiratory support apparatus, the detector is preferably disposed on a downstream side of the bend in the flow path.

Thereby, it is possible to perform appropriate control of the humidifier and a protection operation of the blower even in a flow path configuration with a long flow path length.

Further, the respiratory support apparatus preferably further includes a flow sensor that is disposed on the side of the blower with respect to the intermediate position between the intake port and the blower in the flow path, and the detector is preferably disposed on a downstream side with respect to an upstream-side measurement point of the flow sensor.

Thereby, it is possible to perform appropriate control of the humidifier and a protection operation of the blower even in a flow path configuration with a long flow path length.

Further, the respiratory support apparatus preferably further includes a first controller that controls an operation of the humidifier based on the temperature detected by the detector and a humidity of the airflow, where the humidity is detected by the detector, and the first controller controls the operation of the humidifier such that the temperature of the airflow and the humidity of the airflow approach a target temperature and a target humidity, respectively, where the airflow is discharged from the exhaust port.

Thereby, it is possible to appropriately control the humidifier such that the airflow to be delivered from the humidifier to a patient becomes airflow suitable for the patient's breathing.

Further, the respiratory support apparatus preferably further includes a second controller that controls an operation of the humidifier based on the temperature detected by the detector, and the second controller preferably limits or stops the operation of the humidifier in a case where the temperature detected by the detector exceeds a first threshold temperature.

Thereby, it is possible to prevent the lifespan of the blower from being shortened and to prevent the probability of failure of the blower from increasing.

Further, in the respiratory support apparatus, the second controller preferably causes the operation of the humidifier to resume in a case where the second controller limits or stops the operation of the humidifier and then the temperature detected by the detector has decreased to a second threshold temperature or lower, where the second threshold temperature is equal to or lower than the first threshold temperature.

Thereby, it is possible to continuously use the respiratory support apparatus in a state in which the temperature of the blower is equal to or lower than the heat-resistant temperature.

Further, the respiratory support apparatus is preferably applied to a CPAP apparatus.

Thereby, it is possible to realize the respiratory support apparatus in a more preferable manner.

According to the respiratory support apparatus of the present invention, it is possible to attempt optimization of the temperature and humidity control of airflow to be supplied from a humidifier to a patient and optimization of the temperature management in the apparatus.

### REFERENCE SIGNS LIST

1 Patient
10 Mask
20 Tube
100 CPAP apparatus
100A Main body housing
110 Accommodation case
111 Operation panel
112 Tube connector
113 Intake port
114 Power connector
120 Circuit board
122 Controller
123 Heating controller
124 Respiratory waveform analyzer
125 Communicator
130 Flow path case
131 Blower
132 Flow path
132a Guidance path
132b Blower disposition chamber
132aa to 132ad Bend
150 Base
151 Water tank
152 Lid
152a Air inlet port
152b Air exhaust port
153, 169 Heater
154 AC/AD converter
155 Sheet metal member
156 Fan
150A Humidifier
162 Temperature/humidity sensor (detector)
164 Flow sensor
165 Pressure sensor
166 Temperature sensor

1. A respiratory support apparatus, comprising:
a main body housing that includes an intake port and an exhaust port and forms a flow path of air from the intake port to the exhaust port;
a blower that is disposed in the flow path and generates airflow to be delivered into an airway of a patient;
a humidifier that is disposed on a downstream side of the blower in the flow path, adds moisture to the airflow, and adjusts a temperature of the airflow; and
a detector that is disposed on a side of the blower with respect to an intermediate position between the intake port and the blower in the flow path and detects the temperature of the airflow.

2. The respiratory support apparatus according to claim 1, wherein
the flow path includes a bend that guides the air from the intake port to a position of the blower such that the air largely detours in the main body housing.

3. The respiratory support apparatus according to claim 2, wherein
the detector is disposed on a downstream side of the bend in the flow path.

4. The respiratory support apparatus according to claim 1, further comprising a flow sensor that is disposed on the side of the blower with respect to the intermediate position between the intake port and the blower in the flow path, wherein
the detector is disposed on a downstream side of an upstream-side measurement point of the flow sensor.

5. The respiratory support apparatus according to claim 1, further comprising a first controller that controls an operation of the humidifier based on the temperature detected by the detector and a humidity of the airflow, the humidity being detected by the detector, wherein
the first controller controls the operation of the humidifier such that the temperature of the airflow and the humidity of the airflow approach a target temperature and a target humidity, respectively, the airflow being discharged through the exhaust port.

6. The respiratory support apparatus according to claim 1, further comprising a second controller that controls an operation of the humidifier based on the temperature detected by the detector, wherein
the second controller limits or stops the operation of the humidifier in a case where the temperature detected by the detector exceeds a first threshold temperature.

7. The respiratory support apparatus according to claim 6, wherein
the second controller causes the operation of the humidifier to resume in a case where the second controller limits or stops the operation of the humidifier and then the temperature detected by the detector has decreased to a second threshold temperature or lower, the second threshold temperature being equal to or lower than the first threshold temperature.

8. The respiratory support apparatus according to claim 1, which is applied to a continuous positive airway pressure (CPAP) apparatus.
